(19) United States
(12) Patent Application Publication
(10) Pub. No.: US 2026/0224290 A1
(43) Pub. Date: Aug. 6, 2026

(54) ELECTROMAGNETIC (EM) MEDICAL DEVICE INSTRUMENT TRACKING APPARATUS

(71) Applicant: GE Precision Healthcare LLC, Waukesha, WI (US)

(72) Inventors: Jacob Olesen, Copenhagen (DK); Fredrik Gran, Limhamn (SE); Henrik Jensen, Charlottenlund (DK)

(73) Assignee: GE Precision Healthcare LLC, Waukesha, WI (US)

(21) Appl. No.: 19/043,584

(22) Filed: Feb. 3, 2025

(51) Int. Cl. A61B 34/20

(52) U.S. Cl. CPC A61B 34/20; A61B 2034/2051

(57) ABSTRACT

An electromagnetic medical device instrument tracking apparatus includes an elongated hollow channel. The elongated hollow channel includes a cylindrically shaped body configured to receive elongated shafts of medical devices having instruments at ends of the elongated shafts, a distal end, an inlet at the distal end, a proximal end, and an outlet at proximal end. The elongated hollow channel further includes a fastener coupled at the distal end. The elongated hollow channel further includes at least one electromagnetic sensor disposed at the proximal end. The elongated hollow channel further includes an electrical pathway in electrical communication with the at least one electromagnetic sensor and configured to route signals from the at least one electromagnetic sensor off the apparatus The cylindrically shaped body positions the electromagnetic sensor by an instrument located at an end of an elongated shaft of a medical device installed in the cylindrically shaped body.

### FIELD

The following generally relates to ultrasound imaging, and finds particular application to an electromagnetic (EM) medical device instrument (e.g., a biopsy needle, an ablation catheter, etc.) tracking apparatus.

### BACKGROUND

Ultrasound imaging provides real-time imaging of information about the interior of a subject such as tissue, organs, etc. An example ultrasound imaging system generally includes an ultrasound imaging probe and a console. The ultrasound probe houses a transducer array, and the console includes or is in electrical communication with a display monitor and a user interface. The transducer array transmits a pressure wave and receives echoes produced in response to the pressure wave interacting with structure such as tissue, blood cells, etc. The echoes are converted to analog signals, which are amplified, digitized, and beamformed to produce scan lines of radio frequency (RF) data. The scan lines are processed (e.g., band-pass filtering, envelope detection, logarithmic compression, etc.), scan converted, and displayed as a 2-D (B-mode) ultrasound image.

Some ultrasound imaging probes are configured for procedures performed in a cavity of the body, e.g., through small incisions to guide advancement of an instrument carried at a proximal end of a medical device to target tissue of interest for a procedure. An example of such an ultrasound imaging probe includes a handle, an elongated shaft, and a transducer array disposed at a proximal end of the shaft. An example of such a medical device includes a handle, an elongated shaft, and an instrument (e.g., a blade, a needle, scissors, an ablation catheter, etc.) for the procedure disposed at a proximal end of the shaft. An example of such a procedure includes an image-guided laparoscopic procedure such as an image-guided biopsy, ablation, etc. procedure.

In general, the elongated shaft of the ultrasound imaging probe is advanced through the incision and into the cavity and employed to localize the target tissue of interest. The elongated shaft of the medical device is also advanced through a different incision and guided to the target tissue of interest using ultrasound images. A tracking system tracks the spatial location of the ultrasound imaging probe and the medical device in three-dimensional (3-D) space, registers the coordinate systems of the ultrasound imaging system and the medical device, and superimposes graphical indicia representing the instrument over an ultrasound image generated with ultrasound data acquired with the ultrasound imaging probe and at an estimated location of the instrument with respect to the target tissue of interest in the ultrasound image.

In one instance, the tracking system includes an electromagnetic (EM) tracking system that detects signals from EM sensors employed with the ultrasound imaging probe and the medical device. In some instances, an external EM sensor is attached to a distal end of the elongated shaft of the medical device nearer to or in a region where the elongated shaft couples with the handle, and the signal from the EM sensor is utilized to estimate a spatial orientation of the instrument. Such placement of the EM sensor assumes a rigid elongated shaft where the spatial orientation of the instrument can be derived by adding the distance between the EM sensor and the instrument to the tracked location of the EM sensor at the coupling to the handle. However, the elongated shaft is not rigid and is susceptible to deflect. Unfortunately, deflection may result in an inaccurate estimation of the spatial orientation of the instrument. Even without deflection, angular errors accumulate over the distance between the EM sensor and the instrument, leading to an inaccurate estimation of the spatial orientation of the instrument.

In another instance, an EM sensor is physically integrated in and part of the elongated shaft of the medical device, e.g., near the tip of the medical device. Unfortunately, this ties the tracking to a specific technology provider, i.e., the technology provider of the medical device. In addition, the ultrasound imaging probe and the medical device usually originate from two different technology providers, and the coordinate systems of the ultrasound imaging probe and the medical device need to be registered to map the instrument (and optionally the elongated shaft) to the ultrasound image generated by the ultrasound imaging system. This further ties the tracking to the specific technology provider, i.e., an EM tracking platform of the technology provider. Furthermore, the particular medical device may not be the best or desired medical device for the procedure.

In view of at least the foregoing, there is an unresolved need for an improved approach for EM-based tracking of an instrument of a medical device for an ultrasound image-guided procedure and/or other procedure.

### SUMMARY

Aspects of the application address the above matters, and others. This summary introduces concepts that are described in more detail in the detailed description. It should not be used to identify essential features of the claimed subject matter, nor to limit the scope of the claimed subject matter.

In one aspect, an electromagnetic medical device instrument tracking apparatus includes an elongated hollow channel. The elongated hollow channel includes a cylindrically shaped body configured to receive elongated shafts of medical devices having instruments at ends of the elongated shafts, a distal end, an inlet at the distal end, a proximal end, and an outlet at proximal end. The elongated hollow channel further includes a fastener coupled at the distal end. The elongated hollow channel further includes at least one electromagnetic sensor disposed at the proximal end. The elongated hollow channel further includes an electrical pathway in electrical communication with the at least one electromagnetic sensor and configured to route signals from the at least one electromagnetic sensor off the apparatus The cylindrically shaped body positions the electromagnetic sensor by an instrument located at an end of an elongated shaft of a medical device installed in the cylindrically shaped body.

In another aspect, a method includes selecting a medical device for a procedure, wherein the medical device includes an elongated shaft carrying an instrument at a proximal end. The method further includes selecting an EM medical device instrument tracking apparatus corresponding to the selected medical device. The EM medical device instrument tracking apparatus includes an elongated hollow channel. The elongated hollow channel includes a cylindrically shaped body configured to receive elongated shafts of medical devices having instruments at ends of the elongated shafts, a distal end, an inlet at the distal end, a proximal end; and an outlet at proximal end. The elongated hollow channel further includes a fastener coupled at the distal end. The elongated hollow channel further includes at least one electromagnetic sensor disposed at the proximal end. The elongated hollow channel further includes an electrical pathway in electrical communication with the at least one electromagnetic sensor and configured to route signals from the at least one electromagnetic sensor off the apparatus. The method further includes installing the selected medical device in the EM medical device instrument tracking apparatus. The cylindrically shaped body positions the electromagnetic sensor by an instrument located at an end of an elongated shaft of a medical device installed in the cylindrically shaped body. The method further includes fastening the selected medical device and the EM medical device instrument tracking apparatus together. The method further includes connecting the electrical pathway of the EM medical device instrument tracking apparatus to the tracking system.

In another aspect, an electromagnetic medical device instrument tracking apparatus includes an elongated hollow channel. The elongated hollow channel includes a proximal end. The electromagnetic medical device instrument tracking apparatus further includes at least one electromagnetic sensor disposed at the proximal end of the elongated hollow channel. The electromagnetic medical device instrument tracking apparatus further includes an electrical pathway in electrical communication with the at least one electromagnetic sensor.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example that includes at least a medical device with an instrument and an apparatus for placement of a tracking sensor near the instrument, in accordance with an aspect of an embodiment(s) herein.

FIG. 2 schematically illustrates a non-limiting example of a side view of the apparatus for placement of the tracking sensor near the instrument, in accordance with an aspect of an embodiment(s) herein.

FIG. 3 schematically illustrates a non-limiting example of a perspective view of the apparatus for placement of the tracking sensor near the instrument, in accordance with an aspect of an embodiment(s) herein.

FIG. 4A schematically illustrates a prior art medical device, in accordance with an aspect of an embodiment(s) herein.

FIG. 4B schematically illustrates another prior art medical device, in accordance with an aspect of an embodiment(s) herein.

FIG. 5 schematically illustrates a non-limiting example of the medical device installed in the apparatus for placement of the tracking sensor near the instrument, in accordance with an aspect of an embodiment(s) herein.

FIG. 6 schematically illustrates another non-limiting example of a side view of the apparatus for placement of the tracking sensor near the instrument, in accordance with an aspect of an embodiment(s) herein.

FIG. 7 schematically illustrates yet another non-limiting example of a side view of the apparatus for placement of the tracking sensor near the instrument, in accordance with an aspect of an embodiment(s) herein.

FIG. 8 schematically illustrates still another non-limiting example of a side view of the apparatus for placement of the tracking sensor near the instrument, in accordance with an aspect of an embodiment(s) herein.

FIG. 9 schematically illustrates still yet another non-limiting example of a side view of the apparatus for placement of the tracking sensor near the instrument, in accordance with an aspect of an embodiment(s) herein.

FIG. 10 schematically illustrates still yet another non-limiting example of a side view of the apparatus for placement of the tracking sensor near the instrument, in accordance with an aspect of an embodiment(s) herein.

FIG. 11 schematically illustrates a non-limiting example of a set of medical devices, in accordance with an aspect of an embodiment(s) herein.

FIG. 12 schematically illustrates a non-limiting example of a set of the apparatuses for placement of the tracking sensor near the instrument, in accordance with an aspect of an embodiment(s) herein.

FIG. 13 illustrates a non-limiting example of the medical devices of the set of medical devices of FIG. 11 installed in the set of the apparatuses for placement of the tracking sensor near the instrument of FIG. 12, in accordance with an embodiment(s) herein.

FIG. 14 schematically illustrates another non-limiting example of the medical device installed in the apparatus for placement of the tracking sensor near the instrument, in accordance with an aspect of an embodiment(s) herein.

FIG. 15 schematically illustrates yet another non-limiting example of the medical device installed in the apparatus for placement of the tracking sensor near the instrument, in accordance with an aspect of an embodiment(s) herein.

FIG. 16 illustrates a non-limiting example of a flow chart for a method of employing the apparatus for placement of the tracking sensor near the instrument with the medical device, in accordance with an embodiment(s) herein.

### DETAILED DESCRIPTION

Embodiments of the present disclosure will now be described, by way of example, with reference to the figures, in which a system and/or a method includes an electromagnetic (EM) medical device instrument tracking apparatus. In one instance the EM medical device instrument tracking apparatus at least includes an elongated hollow channel configured to receive an elongated shaft of a medical device carrying an instrument (e.g., a biopsy needle, an ablation catheter, etc.) of the medical device, at least one EM sensor disposed nearer a proximate end of the elongated hollow channel (relative to the opposing distal end of the elongated hollow channel), an electrical pathway configured to route signals from the EM sensor off the EM medical device instrument tracking apparatus, and a fastening mechanism for fastening the EM medical device instrument tracking apparatus to the medical device.

As previously discussed, with existing approaches, an external EM sensor is attached to a distal end of the elongated shaft of the medical device nearer to or in a region where the elongated shaft couples with the handle and away from the instrument, and the signal from the EM sensor is utilized to estimate a spatial orientation of the instrument, which may result in an inaccurate estimation of the spatial orientation of the instrument, e.g., due to an accumulation of angular errors and/or deflection of the elongated shaft, and, hence, the instrument. Alternatively, the EM sensor is physically integrated in the elongated shaft, near the tip of the medical device, which, unfortunately, ties tracking to a specific technology provider, i.e., the technology provider of the medical device and the EM tracking platform of the technology provider, and the medical device may not be the best or desired medical device for the procedure.

The approach described herein mitigates such shortcomings of the aforementioned existing approaches and/or other shortcomings. As described in greater detail below, the EM medical device instrument tracking apparatus is separate from the medical device and includes at least one EM sensor. The medical device is installed in and/or received by the EM medical device instrument tracking apparatus, which positions the at least one EM sensor near the proximate end of the elongated hollow channel, and hence, the instrument carried by the elongated shaft of the medical device. With this approach, tracking is not tied to any particular medical device and/or technology provider of the medical device and is not subject to inaccurate estimates due to angular errors and the distance between the EM sensor and the instrument and/or deflection of the elongated shaft of the medical device carrying the instrument.

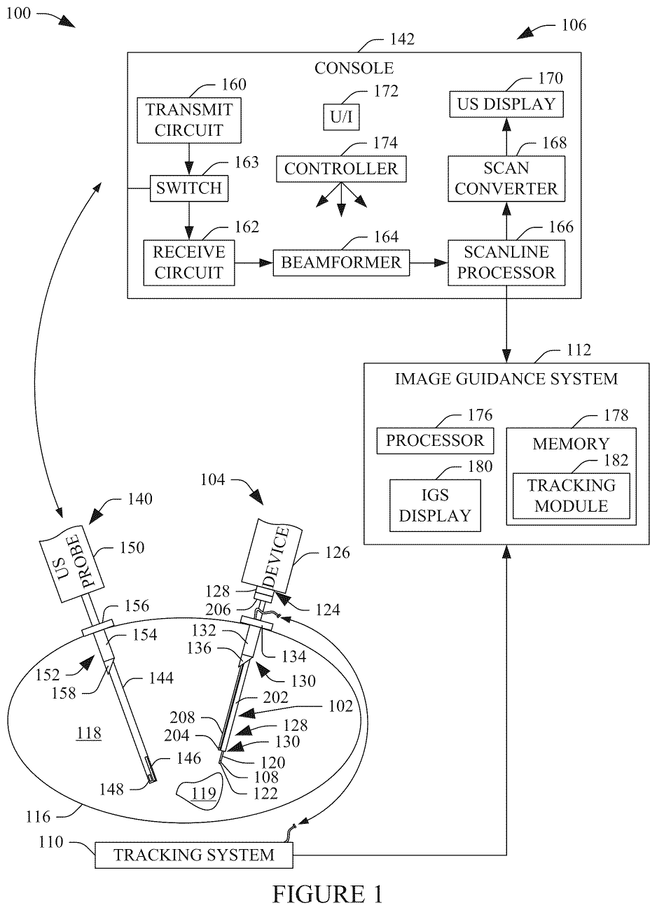

FIG. 1 schematically illustrates a system 100 that includes an EM medical device instrument tracking apparatus 102 in connection with a medical device 104 configured for at least an ablation procedure (e.g., includes an ablation catheter, etc.), a biopsy procedure (e.g., includes a blade, a needle, scissors, etc.), an ultrasound imaging system 106, a tracking system 110, and an image guidance system 112. For explanatory purposes, the system 100 is illustrated in connection with a subject 116 with a cavity 118 that includes a target of interest 119 for the procedure. Medical devices with other instruments for similar and/or other procedures are also contemplated herein.

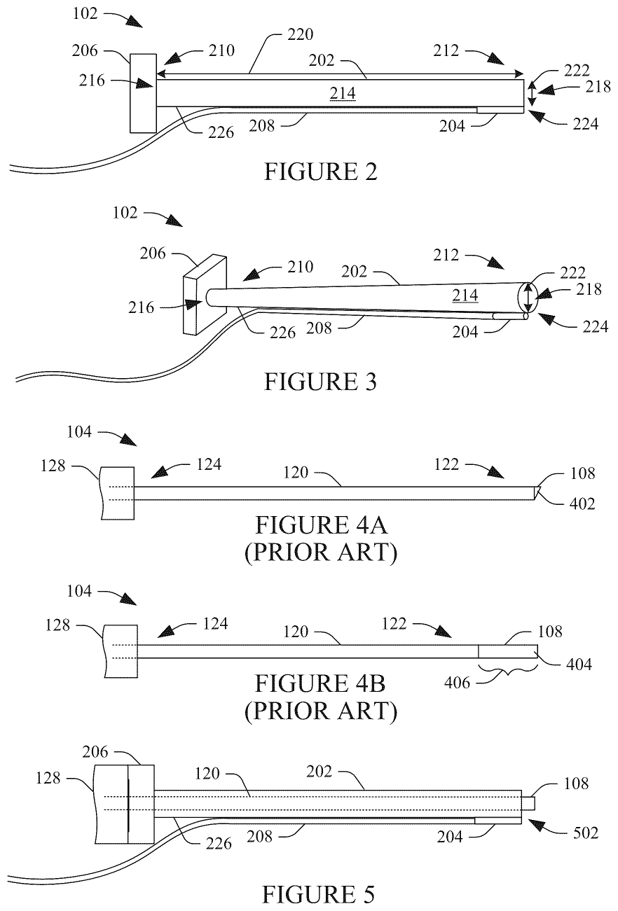

FIGS. 2 and 3 schematically illustrate an example of the EM medical device instrument tracking apparatus 102 without the other components and/or systems illustrated in FIG. 1. FIG. 2 schematically illustrates a side view of the EM medical device instrument tracking apparatus 102, and FIG. 3 schematically illustrates a perspective view of the EM medical device instrument tracking apparatus 102. With reference to FIGS. 1-3, the EM medical device instrument tracking apparatus 102 includes an elongated hollow channel (e.g., a sheath, a canula, etc.) 200, at least one EM sensor 204, a fastener 206, and an electrical pathway 208 for the at least one EM sensor 204 for routing signals from the at least one EM sensor 204 off of the EM medical device instrument tracking apparatus 102.

In this example, the elongated hollow channel 200 includes a cylindrically shaped body with a distal end 210, a proximal end 212, a hollow tubular cavity 214, an inlet 216 at the distal end 210, and an outlet 218 at the proximal end 212. In another instance, the elongated channel 202 includes an elliptical, oval, and/or otherwise shaped body. The cylindrically shaped body 202 has a length 220 and a diameter 222. In general, the length 220 and the diameter 222 are configured based on a gauge (i.e., a diameter) of a shaft of the medical device 104 as the cylindrically shaped body 202 is configured to receive the shaft of the medical device 104. That is, the diameter 222 is large enough to receive the shaft (and possibly one or more other items), while minimizing space between the cylindrically shaped body 202 and the shaft.

The illustrated EM medical device instrument tracking apparatus 102 further includes an EM sensor 204. The EM sensor 204 is disposed at a proximal end region 224 of the cylindrically shaped body 202. In another instance, the EM sensor 204 is disposed with a gap between the EM sensor 204 and the proximal end region 224. Further, the illustrated EM sensor 204 is disposed on an outer surface 226 of the cylindrically shaped body 202. In another instance (and as described below), the EM sensor 204 is otherwise disposed in connection with the cylindrically shaped body 202. Any known technique and/or other approaches for securing the EM sensor 204 to the outer surface 226 is contemplated herein.

In one instance, the EM sensor 204 includes a passive EM sensor. In another instance, the EM sensor 204 includes an active EM sensor. In yet another instance, the EM sensor 204 includes a combination of a passive EM sensor and an active EM sensor. In any instance, the EM sensor 204 includes a coil, an element, and/or other EM sensor technology. In another instance, an inertial tracking device is alternatively or additionally utilized. A suitable EM sensor for determining a spatial orientation of cylindrically shaped body 202 includes at least five (5) degrees of freedom. For example, a five (5) degrees of freedom EM sensor would allow for determining a spatial orientation of cylindrically shaped body 202 about itself. In another instance, the EM sensor includes six (6) degrees of freedom, which allows for determining a spatial orientation of cylindrically shaped body 202 in three-dimensional (3-D) space.

The fastening member 206 is coupled at the distal end 210 of EM medical device instrument tracking apparatus 102. The fastening member 206 is configured to fasten the EM medical device instrument tracking apparatus 102 to the medical device 104. In one instance, the fastening member 206 includes one of two complementary sides configured to engage, where the medical device 104 include the other complementary side (as discussed below). For example, one of the two sides may include external threads and the other of the two sides may include complementary internal threads. Other mechanisms such as a clamp, a screw, etc. are contemplated herein. The illustrated shape of the fastening member 206 is for explanatory purposes and is not limiting.

The electrical pathway 208, similar to the EM sensor 204, is disposed on the outer surface 226 of the cylindrically shaped body 202. The electrical pathway 208 is in electrical communication with the EM sensor 204 and routes signals therefrom off the EM medical device instrument tracking apparatus 102. In the illustrated example, the electrical pathway 208 runs along the outer surface 226 to about the distal end 210 and then extends off of the outer surface 226 and around the fastening member 206. In another instance, the electrical pathway 208 is routed through the fastening member 206. Any known technique and/or other approaches for securing the electrical pathway 208 to the outer surface 226 is contemplated herein.

With reference to FIG. 1, the medical device 104 includes an elongated shaft 120 having a long axis. The elongated shaft 120 carries the instrument 108 at a proximal end 122. An opposing distal end 124 of the elongated shaft 120 is coupled to a handle 126. A fastening member 128 disposed at the opposing distal end 124 acts in conjunction with the fastening member 206 of the EM medical device instrument tracking apparatus 102, e.g., as a second side of complementary sides, etc., for securing or fastening the EM medical device instrument tracking apparatus 102 to the medical device 104.

FIGS. 4A and 4B schematically illustrate example prior art medical devices 104. With reference to FIGS. 1 and 4A, the instrument 108 includes a blade 402 for a biopsy procedure. In another instance, the instrument 108 carries a needle, scissors, etc. With reference to FIGS. 1 and 4B, the instrument 108 includes an ablation catheter 404. The illustrated ablation catheter 404 has an active region 406 that is around one to two centimeters in length. Other size active regions 406 are also contemplated herein. In other instances, the instrument 108 includes a radiofrequency (RF) catheter, a microwave catheter, cryoablation catheter, a laser, and/or other instrument.

With reference to FIG. 1, as described herein, the EM medical device instrument tracking apparatus 102 is configured to receive the elongated shaft of the medical device 104. A magnified view of a portion the medical device 104 and the EM medical device instrument tracking apparatus 102 from FIG. 1 is schematically illustrated in FIG. 5. With reference to FIG. 1-5, in general, the length 220 of the cylindrically shaped body 202 and the location of the EM sensor 204 on the cylindrically shaped body 202 are such that a distance 502 between the EM sensor 204 and the instrument 108 is as short as reasonably possible to the instrument 108. In one instance, the distance 502 is on an order of one centimeter or less.

The actual distance depends on the instrument 108 and/or other factors. In general, the closer the EM sensor 204 is to the instrument 108, the more accurate the tracking will be.

As previously discussed, with existing approaches, an external EM sensor is attached to a distal end of the elongated shaft of the medical device near to or in a region where the elongated shaft couples with the handle, and the signal from the EM sensor is utilized to estimate a spatial orientation of the instrument, where such placement of the EM sensor assumes a rigid elongated shaft and the spatial orientation of the instrument is derived by adding the distance between the EM sensor and the instrument to the tracked location of the EM sensor at the coupling to the handle, and this may result in an inaccurate estimation of the spatial orientation of the instrument, e.g., due to an accumulation of angular errors and/or deflection of the elongated shaft, and, hence, the instrument.

Alternatively, the EM sensor is physically integrated in the elongated shaft, near the tip of the medical device, which ties tracking to a specific technology provider, i.e., the technology provider of the medical device. In addition, the ultrasound imaging probe and the medical device usually originate from two different technology providers, and the ultrasound imaging probe and the medical device need to be registered to a same coordinate system to map the elongated shaft of the medical device to the image generated by the ultrasound imaging system, which further ties tracking to the specific technology provider, i.e., EM tracking platform of the technology provider. In addition, the medical device may not be the best or desired medical device for the procedure.

The approach described herein mitigates such shortcomings and/or other shortcomings of the aforementioned existing approaches. As described in greater detail below, the EM medical device instrument tracking apparatus 102 is separate from the medical device 104 and includes at least one EM sensor 204. The medical device 104 is installed in and/or received by the EM medical device instrument tracking apparatus 102, which places the at least one EM sensor 204 near the proximate end 212 of the cylindrically shaped body 202, and, hence, the instrument 108 carried by the elongated shaft 120 of the medical device 104. With this approach, tracking is not tied to any particular medical device and/or technology provider of the medical device and is not subject to inaccurate estimates due to angular errors and the distance between the EM sensor and the instrument and/or deflection of the elongated shaft of the medical device carrying the instrument.

Returning to FIG. 1, the EM medical device instrument tracking apparatus 102 and the medical device 104 are shown in connection with a trocar 130 in an incision in an outer wall of the subject 116. The illustrated trocar 130 includes a rigid hollow elongated tube 132, a port 134, and a tapered tip 136. The trocar 130 serves as a minimally invasive gateway for the EM medical device instrument tracking apparatus 102, and, thus, the medical device 104 to the target of interest 120 in the cavity 118 of the subject 116.

The ultrasound imaging system 106 includes an ultrasound imaging probe 140 and a console 142. The ultrasound imaging probe 140 and the console 142 interface with each other via a communication channel, which includes wired technology, e.g., complimentary interfaces and a cable therebetween (as illustrated), and/or wireless technology, e.g., Wi-Fi, etc.

The ultrasound imaging probe 140 includes an elongated shaft 144 with opposing ends. A transducer array 146 is disposed at one of the ends. The transducer array 146 includes one or more transducer elements. Examples of suitable arrays include 64, 128, 192, 256, and/or other arrays, including larger and smaller arrays, one dimensional (1-D) or two dimensional (2-D), etc. The transducer array 146 can be linear, curved, and/or otherwise shaped, fully populated, sparse and/or a combination thereof, etc. The transducer array 146 is configured to convert an excitation electrical signal to an ultrasound pressure field and vice versa.

By way of non-limiting example, the one or more transducer elements can be selectively excited via an excitation electrical (pulsed) signal, which causes at least a sub-set of the transducer elements to transmit an ultrasound pressure field into an examination or scan field of view. The ultrasound pressure field may include a focused ultrasound beam, a defocused (spherical) wave, and/or other ultrasound signal. The one or more transducer elements receive echo signals and generate analog electrical signals indicative thereof. The echo signals are generated in response to the transmitted ultrasound pressure field interacting with structure, such as tissue and/or blood cells flowing in a portion of a vessel.

The ultrasound imaging probe 140 further includes a tracking sensor 148. The tracking sensor 148 is located at a same end of the elongated shaft 144 as the transducer array 146. In this example, the tracking sensor 148 is integrated with the ultrasound imaging probe 140. In another instance, the tracking sensor 148 is removably attached to the ultrasound imaging probe 140. An example of the tracking sensor 148 include an EM tracking sensor, an inertial tracking device, etc. In another instance, the tracking sensor 148 is disposed elsewhere in the ultrasound imaging probe 140. A handle 150 is disposed at the other end of the shaft 158.

The ultrasound imaging probe 140 is shown in connection with a trocar 152, which is substantially similar to the trocar 130. The illustrated trocar 152 includes a rigid hollow elongated tube 154, a port 156, and a tapered tip 158. The trocar 152 serves as a minimally invasive gateway for the elongated shaft 144 of the ultrasound imaging probe 140, and, thus, the transducer array 146 and tracking sensor 148. The ultrasound imaging probe 140 is used to guide the instrument 108 to the target of interest 120 in the cavity 118 of the subject 116.

The console 142 includes a transmit circuit 160 configured to generate the excitation electrical signal provided to transducer array 146 for transmitting the ultrasound pressure field. In one instance, this includes generating delays for individual elements of the transducer array 146, e.g., for transmit focusing, beam steering, etc.

The console 142 further includes a receive circuit 162 configured to receive the analog electrical signals. In one instance, the receive circuit 162 is further configured to pre-process the analog electrical signals, e.g., amplify, digitize, focus, and/or otherwise process the analog electrical signals. For example, in one instance the receive circuit 162 includes an amplifier and a corresponding analog to digital converter (ADC) for each element, where each amplifier amplifies a corresponding analog electrical signal from a micro-volt level to a voltage range of the ADC.

The console 142 further includes a switch 163 configured to switch between the transmit circuit 160 and the receive circuit 162, e.g., by electrically connecting the transmit circuit 160 to the transducer array 146 for a transmit operation and electrically connecting the receive circuit 162 to the transducer array 146 for a receive operation. In an alternative instance, separate switches are employed for each of the transmit circuit 160 and the receive circuit 162.

The console 142 further includes a beamformer 164. For receive operations, the beamformer 164 is configured to beamform, e.g., via delay-and-sum (e.g., a matched-filter beamformer, etc.) and/or other beamforming, the signals from the receive circuit 162 and construct a scanplane of scanlines of radiofrequency (RF) data (RF signal) for the echoes for each receive operation. With delay-and-sum beamforming, the digital signal for each element is delayed to align the signals in time, amplified, and then summed. The output of the beamformer 164 includes the RF signal.

The console 142 further includes a scanline processor 166 configured to perform other processing on the data such as filtering (e.g., via a Finite Impulse Response (FIR) filter, an Infinite Impulse Response (IIR) filter, etc.), Time Gain Compensation (TGC), I/Q demodulation, envelope detection, logarithmic compression, noise rejection, and/or other processing, and output frames of data. When configured for I/Q demodulation, the scanline processor 166 down mixes the RF signal and, optionally, apply low pass filtering and/or decimation. This may include employing a Hilbert Transform, a combination of a Complex-Demodulation Band Pass Filter and optional decimation, and/or other processing.

The scanline processor 166 detects and extracts the envelope (e.g., an amplitude) of the I/Q signal (when the scanline processor 166 I/Q demodulates the RF signal) or the RF signal (when the scanline processor 166 does not I/Q demodulate the RF signal). In one instance, this is achieved using a Hilbert Transform and/or other approach. The scanline processor 166 compresses the extracted envelope, reducing the dynamic range thereof, e.g., to reduce the dynamic range to a predetermined display precision by a logarithmic (log)-based dynamic range compression and/or otherwise, and outputs a scanline. The scanline processor 166 outputs the processed scanlines as a frame/image (e.g., a B-mode image).

The console 142 further includes a scan converter 168. The scan converter 168 is configured to scan convert the image into a coordinate system of an ultrasound system (US) display 170. The scan converter 168 can be configured to employ analog and/or digital scan converting techniques. The ultrasound system display 170 is integrated with the console 142. In another instance, ultrasound system display 170 is a separate and/or remote display monitor in electrical communication with the console 142.

The console 142 further includes a user interface (U/I) 172. The user interface 172 includes one or more input devices (such as a button, a knob, a slider, a touch screen, a mouse, a keyboard, etc.) and/or other input device, and/or one or more output devices such as a visible, audible, etc. indicator. The user interface 172 allows a user to control an operation of the ultrasound imaging system 106. For example, in one instance, user interface 172 receives an input indicative of an imaging protocol including tracking of the medical device 104. The user interface 172 is shown integrated with the console 142. In another instance, the user interface 172 is a separate and/or remote keyboard, keypad, touch screen, etc. in electrical communication with the console 142.

The console 142 further includes a controller 174. The controller 174 includes a processor(s) such as a microprocessor (mP), a central processing unit (CPU), a graphics processing unit (GPU), etc., and computer readable storage medium. The computer readable storage medium includes computer readable instructions, and the processor is configured to execute instructions stored in the computer readable storage medium. The controller 174 is configured to control one or more of the transmit circuit 160, the receive circuit 162, the switch 163, the beamformer 164, the scanline processor 166, the scan converter 168, the ultrasound imaging display 170, and the user interface 172. One or more of the components of the console 142 can be implemented in software and/or hardware.

The tracking system 110 can be a passive EM tracking system or an active EM tracking system. In either instance, the tracking system 110 is configured to track a position and an orientation of the medical device 104. With a passive EM tracking system, the tracking system 110 generates a defined external electromagnetic field. The EM sensor 204 is configured to generate signals in response to excitation by the external electromagnetic field. The signal is routed via the electrical pathway 208 along the side of the cylindrical body 204 to the tracking system 110. The tracking system 110 processes the signal to determine the position and orientation of the EM sensor 204 in real-time. With an active EM tracking system, as the medical instrument 108 moves in the electromagnetic field, the EM sensor 204 generate signals, which are detected and processed to continuously track the position and orientation of the medical device 104 in real-time.

The image guidance system 112 includes at least a processor 176 (e.g., a central processing unit, a microprocessor, etc.), a memory 178 (i.e., computer readable medium, which includes physical memory, etc., and excludes transitory medium such as a signal, carrier wave or other transitory medium), and an image guided system (IGS) display 180. The memory 178 includes computer readable instructions, and the processor 176 is configured to execute instructions stored in the memory 178. The image guided system display 180 is shown integrated with the image guidance system 112. In another instance, the image guided system display 180 is a separate and/or remote display monitor in electrical communication with the image guidance system 112.

The memory 178 includes a tracking module 182 configured to process the location and/or direction information from the tracking system 110 and image from the scanline processor 166 of the ultrasound imaging system 106. In one instance, where the instrument 108 is in a plane of the ultrasound beam, the tracking module 182 displays the image with graphical indicia at an estimated location of at least the instrument 108 superimposed or overlaid over the image, providing real-time display of images and the estimated location of at least the instrument 108. An example of suitable indicia includes a crosshair at an estimated location of a tip of the instrument 108. Another example additionally includes an end portion of the elongated shaft 120 along with the instrument 108.

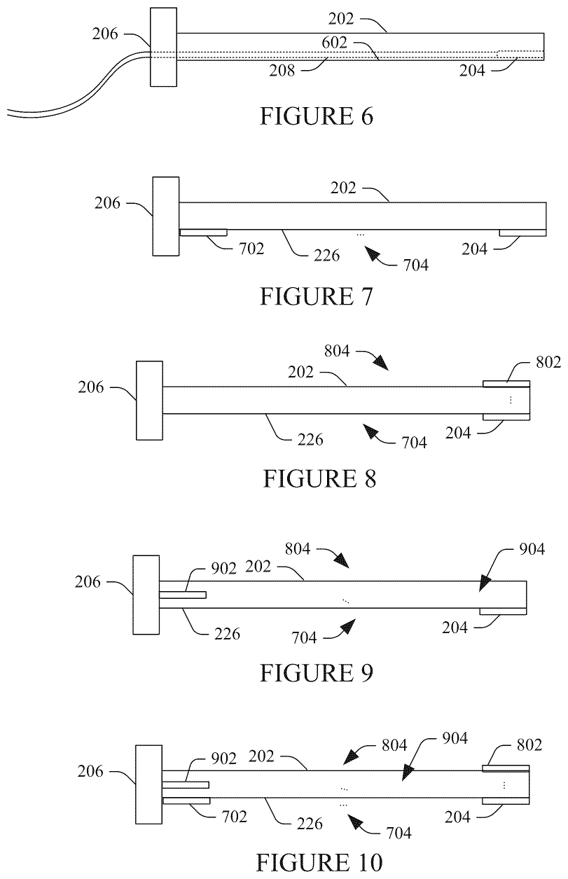

In FIGS. 1-3 and 5, the EM sensor 204 and the electrical pathway 208 are disposed on the outer surface 226 of the cylindrical shaped body 202. FIG. 6 schematically illustrates another example of the EM medical device instrument tracking apparatus 102 in which the EM sensor 204 and the electrical pathway 208 are disposed inside of the cylindrically shaped body 202. In this example, the EM sensor 204 and the electrical pathway 208 are on an inner surface 602 of the cylindrical shaped body 202. In this example, the diameter of the cylindrical shaped body 202 is configured to include enough space at least for the elongated shaft of the medical device 104, the EM sensor 204, and the electrical pathway 208. Any known technique and/or other approaches for securing the EM sensor 204 to the inner surface 602 is contemplated herein.

In FIGS. 1-3 and 5, the EM medical device instrument tracking apparatus 102 include a single sensor, the EM sensor 204 near the tip 132. FIGS. 7, 8, 9 and 10 schematically illustrate variations in which the EM medical device instrument tracking apparatus 102 includes at least two of the EM sensors 204. FIG. 7 depicts an example with two or more of the EM sensors 204 arranged along a same side between ends of the cylindrical shaped body 202. FIG. 8 depicts an example with two or more of the EM sensors 204 arranged along different sides at a same end of the cylindrical shaped body 202. FIG. 9 depicts an example with two or more of the EM sensors 204 arranged along different sides and not at the same end of the cylindrical shaped body 202. FIG. 10 depicts a combination of the examples described in connection with FIGS. 7, 8 and 9.

In FIGS. 7, 8, 9 and 10, the electrical pathway 208 is not shown for clarity and explanatory purposes. However, it is to be appreciated that in one instance a single electrical pathway 208 may be utilized with multiple EM sensors 204, while in another instance, multiple electrical pathways 208 may be utilized with multiple EM sensors 204, including one to one, one to many, etc. combinations. For example, in one instance multiple EM sensors 204 are included on a single integrated chip (IC) and a single electrical pathway 208 routes signals from the multiple EM sensors 204 off the EM medical device instrument tracking apparatus 102. For example, the IC may include two EM sensors, each with five (5) degrees of freedom, and a single electrical pathway 208.

Initially referring to FIG. 7, the EM medical device instrument tracking apparatus 102 includes the EM sensor 204, . . . , and an EM sensor 702. The EM sensor 204, . . . , and the EM sensor 702 are both located on the outer surface 226 of the cylindrical shaped body 202, along a straight line between the ends of the cylindrical shaped body 202. In this example, the EM sensor 204 is located as close as reasonably possible to the end, as described herein. The EM sensor 702 is located at the opposing end of the cylindrical shaped body 202, near the fastener 206. Other EM sensors would be located therebetween. In another instance, at least one of the EM sensors 204 and the EM sensor 702 is located with a space therebetween and at the end. In one instance, the EM medical device instrument tracking apparatus 102 includes only two sensors, the EM sensor 204 and the EM sensor 702, spaced apart. In general, employing two such EM sensors may improve an accuracy of the estimated spatial orientation of the instrument 108, relative to a single EM sensor.

Moving to FIG. 8, the EM medical device instrument tracking apparatus 102 includes the EM sensor 204, . . . , and an EM sensor 802. The EM sensor 204, . . . , and the EM sensor 702 are arranged around a perimeter at the cylindrical shaped body 202, on opposing sides of the cylindrical shaped body 202. The EM sensor 204 is located on the side 704. The EM sensor 802 is located opposite the EM sensor 204, on a side 802 opposite of the side 704. In this example, where there are other EM sensors, they would be located between the EM sensor 204 and the EM sensor 802 around the perimeter at the end 128 of the cylindrical shaped body 202. In a variation, at least one of the EM sensors 204 and/or 802 is disposed with a space between the sensor and the end of the cylindrical shaped body 202.

Moving to FIG. 9, the EM medical device instrument tracking apparatus 102 includes the EM sensor 204, . . . , and an EM sensor 902. The EM sensor 204, . . . , and the EM sensor 902 are arranged on different sides and different ends of the cylindrical shaped body 202. The EM sensor 204 is located on the side 704. The EM sensor 902 is located on a side 904, which is between the side 704 and the side 804. In this example, where there are other EM sensors, they would be likewise located between the side 704 and the side 804, on and/or off of the side 904. In a variation, at least one of the EM sensors 204 and/or 902 is disposed with a space between the EM sensor and the end of the cylindrical shaped body 202.

FIG. 10 depicts an example that is a combination of the examples described in connection with FIGS. 7, 8 and/or 9. For example, in FIG. 10, the EM medical device instrument tracking apparatus 102 includes the EM sensor 204 and two or more of the EM sensors 702, 802 and/or 902. Another variation includes one of the examples discussed in connection with FIGS. 7, 8 and 9 or a combination thereof, but with at least one of the sensors (e.g., the EM sensor 204, the EM sensor 702, the EM sensor 802, the EM sensor 902, etc.) disposed on the inner surface 226 of the cylindrical body 124 202, e.g., as discussed in connection with the example of FIG. 6 and/or otherwise.

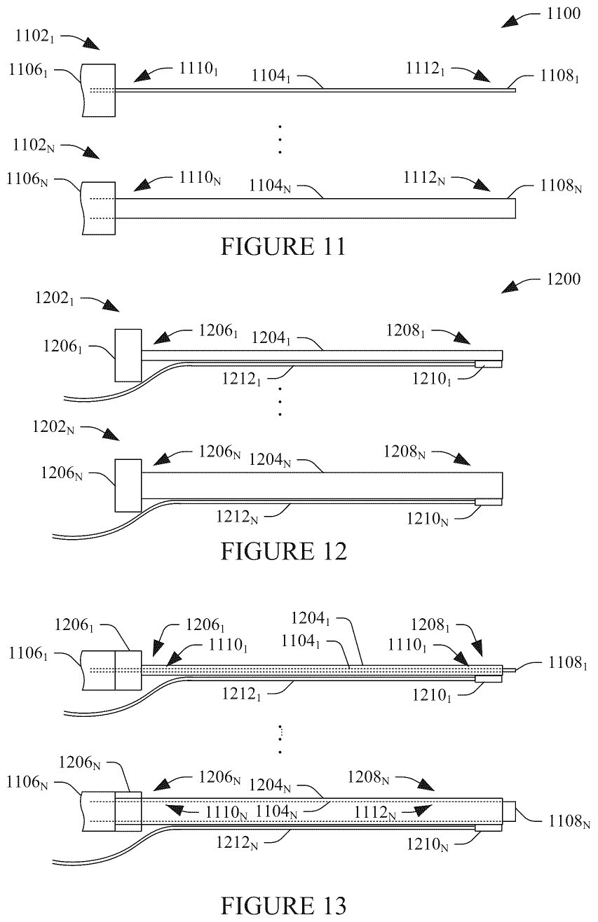

FIGS. 11, 12 and 13 schematically illustrates a plurality of the EM medical device instrument tracking apparatuses 102 in conjunction with a plurality of the medical devices 104. FIG. 11 schematically illustrates a set of the medical devices 104. FIG. 12 schematically illustrates a set of the EM medical device instrument tracking apparatuses 102. FIG. 13 schematically illustrates the set of the medical devices 104 described in connection with FIG. 11 installed in the set of the EM medical device instrument tracking apparatuses 102 described in connection with FIG. 12.

Initially referring to FIG. 11, a set 1100 of the medical devices 104 includes a medical device 1102₁, . . . , and a medical device 1102_{N}, (collectively referred to herein as medical devices 1102), where N is an integer equal to or greater than one.

The medical device 1102₁ includes an elongated shaft 1104₁, a fastening member 1106₁, and an instrument 1108₁. The elongated shaft 1104₁ has a first end 1110₁ and an opposing second end 1112₁. The fastening member 1106₁ is coupled to the first end 1110₁. The instrument 1108₁ is coupled to the opposing second end 1112₁. The instrument 1108₁ can include a biopsy needle, an ablation catheter, etc.

The medical device 1102_{N} includes an elongated shaft 1104_{N}, a fastening member 1106_{N}, and an instrument 1108_{N}. The elongated shaft 1104_{N} has a first end 1110_{N} and an opposing second end 1112_{N}. The fastening member 1106_{N} is coupled to the first end 1110_{N}. The instrument 1108_{N} is coupled to the opposing second end 1112_{N}. The instrument 1108_{N} can include a biopsy needle, an ablation catheter, etc.

The illustrated medical device 1102₁ and the illustrated medical device 1102_{N} are substantially similar except that a diameter of the elongated shaft 1104₁ of medical device 1102₁ is smaller than a diameter of the elongated shaft 1104_{N} of medical device 1102_{N}. Medical devices between the medical device 1102₁ and the medical device 1102_{N} have other diameters. For example, their diameters are between the diameters of the elongated shaft 1104₁ and the elongated shaft 1104_{N}. In another instance, other attributes (e.g., length, instrument, etc.) of the medical devices 1102 are different.

Moving to FIG. 12, a set of the EM medical device instrument tracking apparatuses 1200 includes an EM medical device instrument tracking apparatus 1202₁, . . . , and an EM medical device instrument tracking apparatus 1202_{N}, (collectively referred to herein as EM medical device instrument tracking apparatuses 1202). The EM medical device instrument tracking apparatus 1202₁ includes a cylindrical shaped body 1204₁ with a distal end 1206₁, a proximal end 1208₁, an EM sensor 1210₁, and an electrical pathway 1212₁. The EM medical device instrument tracking apparatus 1202_{N} includes a cylindrical shaped body 1204_{N} with a distal end 1206_{N}, a proximal end 1208_{N}, an EM sensor 1210_{N}, and an electrical pathway 1212_{N}.

The illustrated EM medical device instrument tracking apparatus 1202₁ and the illustrated medical device EM medical device instrument tracking apparatus 1202_{N} are substantially similar except that a diameter of the cylindrical shaped body 1204₁ of the EM medical device instrument tracking apparatus 1202₁ is smaller than a diameter of the cylindrical shaped body 1204_{N} of the EM medical device instrument tracking apparatus 1202_{N}. EM medical device instrument tracking apparatuses between the EM medical device instrument tracking apparatus 1202₁ and the EM medical device instrument tracking apparatus 1202_{N} have other diameters. For example, their diameters are between the diameters of the cylindrical shaped body 1204₁ and the cylindrical shaped body 1204_{N}. In other instance, other attributes (e.g., length, etc.) of the apparatuses 1202 are different.

Turning to FIG. 13, the set of the medical devices 1100 are respectively installed in the set of the EM medical device instrument tracking apparatus 1200. The shaft 1104₁ of the medical device 1102₁ is inside of the cylindrical shaped body 1204₁ of the EM medical device instrument tracking apparatus 1202₁. The fastening member 1106₁ of the medical device 1102₁ is engaged with the fastening member 1204_{N} of the EM medical device instrument tracking apparatus 1202₁. The shaft 1104_{N} of the medical device 1102_{N} is inside of the cylindrical shaped body 1204_{N} of the EM medical device instrument tracking apparatus 1202_{N}. The fastening member 1206_{N} of the medical device 1102_{N} is engaged with the fastening member 1206_{N} of the EM medical device instrument tracking apparatus 1202_{N}.

In FIGS. 11, 12 and 13, each of the EM medical device instrument tracking apparatuses 1202₁, . . . , 1202_{N} is configured based on a corresponding one of the medical devices 1102₁, . . . , 1102_{N} in that a diameter of the cylindrical shaped bodies 1204₁, . . . , 1204_{N} of the EM medical device instrument tracking apparatuses 1202₁, . . . , 1202_{N} corresponds to a diameter of the shafts 1104₁, . . . , 1104_{N} of the medical devices 1102₁, . . . , 1102_{N}. For example, in one instance each of the cylindrical shaped bodies 1204₁, . . . , 1204_{N} of the EM medical device instrument tracking apparatuses 1202₁, . . . , 1202_{N} is configured to minimize or optimize a gap between the cylindrical shaped bodies 1204₁, . . . , 1204_{N} of the EM medical device instrument tracking apparatuses 1202₁, . . . , 1202_{N} and the shafts 1104₁, . . . , 1104_{N} of the medical devices 1102₁, . . . , 1102_{N} so that the EM sensors 1210₁, . . . , 1210_{N} more accurately tracks the instruments 1108₁, . . . , 1108_{N} relative to a larger gap where the sensors 1202₁, . . . , 1202_{N} are further away from the instruments 1108₁, . . . , 1108_{N}.

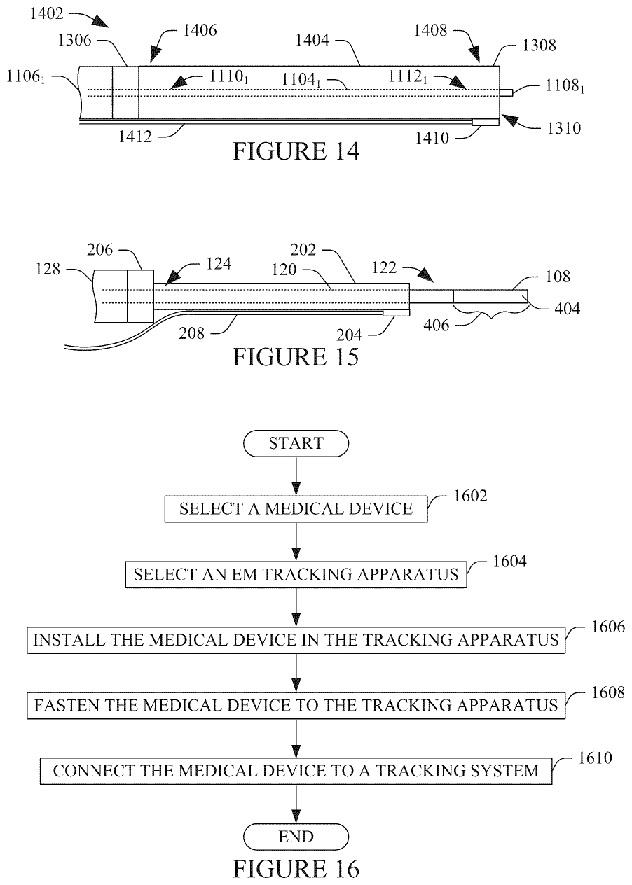

FIG. 14 schematically illustrates another example in which the EM medical device instrument tracking apparatus 102 is configured to receive any of a set of different sized medical devices 104 such as the medical devices 1102₁, . . . , 1102_{N} described in connection with FIGS. 11 and 13. A EM medical device instrument tracking apparatus 1402 includes a cylindrical shaped body 1404 with a distal end 1406, a proximal end 1408, an EM sensor 1410, and an electrical pathway 1412. In this example, a diameter of the cylindrical shaped body 1404 is larger than a largest diameter of the shafts 1104₁, . . . , 1104_{N} of the set of the medical devices 1102₁, . . . , 1102_{N} described in connection with FIGS. 11 and 13. As such, the EM medical device instrument tracking apparatus 1402 can receive any of the medical devices 1102₁, . . . , 1102_{N}. The illustrated embodiment shows the EM medical device instrument tracking apparatus 1402 with the medical devices 1102₁.

FIG. 15 schematically illustrates another example in which the EM medical device instrument tracking apparatus 102. As discussed herein, the closer the EM sensor 204 (FIG. 1) is to the instrument 108 (FIG. 1), the more accurate is the tracking of the instrument 108. As shown in FIGS. 4A and 4B, different instruments 108 may have different sizes, such as a length along the axis of the instruments 108. For example, in FIG. 4A, the instrument 108 (a blade, a needle, scissors, etc.) can be considered at the end of the shaft 120, whereas in FIG. 4B, the instrument 108 (an ablation catheter, etc.) may extend a few centimeters along the length of the shaft 120. As such, different EM medical device instrument tracking apparatuses 104 may have different lengths. FIG. 15 depicts the EM medical device instrument tracking apparatus 102 configured for medical device 104 with the instrument 108 that extends a few centimeters along the length of the shaft 120 such as the ablation instrument discussed in connection with FIG. 4B.

FIG. 16 illustrates a non-limiting example of a flow chart for a method employing the apparatus for placement of the tracking sensor near the instrument with the medical device. It is to be appreciated that the ordering of the acts in the method is not limiting. As such, other orderings are contemplated herein. In addition, one or more acts may be omitted, and/or one or more additional acts may be included.

At 1602, the medical device 106 for a procedure is selected. For example, the medical device 104 is selected based on the type of procedure, etc. In one instance, this includes selecting a particular gauge associated with the medical device 104. At 1604, the EM medical device instrument tracking apparatus 102 corresponding to the selected medical device 106 is selected. In one instance, this includes selecting the EM medical device instrument tracking apparatus 102 for the gauge of the medical device 106. At 1606, the selected medical device 106 is installed in the EM medical device instrument tracking apparatus 102, as described herein and/or otherwise. At 1608, the EM medical device instrument tracking apparatus 102 is fastened to the selected medical device 106, as described herein and/or otherwise. At 1610, the electrical pathway of the EM medical device instrument tracking apparatus 102 is connected to the tracking system 104.

The medical device 106/EM medical device instrument tracking apparatus 102 combination is then employed in an ultrasound image guided procedure, as described herein and/or otherwise. For instance, the EM medical device instrument tracking apparatus 102 is used to track the advancement of the medical device 106 to tissue of interest, e.g., through presenting an ultrasound image with graphical indicia representing the instrument superimposed thereover at a location determined by the signals from the EM sensor. After the procedure, the medical device 106 is uninstalled from the EM medical device instrument tracking apparatus 102. Where the EM medical device instrument tracking apparatus 102 is disposable, the EM medical device instrument tracking apparatus 102 is disposed. Where the EM medical device instrument tracking apparatus 102 is reusable, the EM medical device instrument tracking apparatus 102 is sanitized and/or disinfected. The EM medical device instrument tracking apparatus 102 can then be re-used for another ultrasound image guided procedure. Where the EM medical device instrument tracking apparatus 102 is disposable, the EM medical device instrument tracking apparatus 102 is disposed.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only and are thus not intended to limit in any way the definition and/or meaning of the term “computer.” The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used in practice as some jurisdictions require them, but this should not be taken to mean that the features in the dependent claims are mutually exclusive.

What is claimed is:

1. An electromagnetic medical device instrument tracking apparatus, comprising:
an elongated hollow channel, including:
a cylindrically shaped body configured to receive elongated shafts of medical devices having instruments at ends of the elongated shafts;
a distal end;
an inlet at the distal end;
a proximal end; and
an outlet at proximal end;
a fastener coupled at the distal end;
at least one electromagnetic sensor disposed at the proximal end; and
an electrical pathway in electrical communication with the at least one electromagnetic sensor and configured to route signals from the at least one electromagnetic sensor off the apparatus,
wherein the cylindrically shaped body positions the electromagnetic sensor by an instrument located at an end of an elongated shaft of a medical device installed in the cylindrically shaped body.

2. The electromagnetic medical device instrument tracking apparatus of claim 1, wherein the cylindrically shaped body has a first length, the elongated shaft has a second length, and the first length is shorter than the second length.

3. The electromagnetic medical device instrument tracking apparatus of claim 2, wherein the first length is on an order of one centimeter or less than the second length.

4. The electromagnetic medical device instrument tracking apparatus of claim 1, wherein the cylindrically shaped body has a first diameter, the elongated shaft has a second diameter, and the first diameter corresponds to the second diameter.

5. The electromagnetic medical device instrument tracking apparatus of claim 4, wherein the cylindrically shaped body has a first diameter, the elongated shaft has a second diameter, and the first diameter is agnostic to a second diameter.

6. The electromagnetic medical device instrument tracking apparatus of claim 1, further comprising:
a second electromagnetic sensor disposed at the distal end.

7. The electromagnetic medical device instrument tracking apparatus of claim 1, wherein the fastener fastens the cylindrically shaped body to the medical device.

8. The electromagnetic medical device instrument tracking apparatus of claim 1, wherein the cylindrically shaped body include an outer surface and the at least one electromagnetic sensor is disposed on the outer surface.

9. The electromagnetic medical device instrument tracking apparatus of claim 1, wherein the cylindrically shaped body include an inner surface and the at least one electromagnetic sensor is disposed on the inner surface.

10. The electromagnetic medical device instrument tracking apparatus of claim 1, wherein the instrument includes an ablation catheter.

11. The electromagnetic medical device instrument tracking apparatus of claim 1, wherein the instrument includes a biopsy blade, needle or scissors.

12. The electromagnetic medical device instrument tracking apparatus of claim 1, wherein the apparatus is disposable.

13. The electromagnetic medical device instrument tracking apparatus of claim 1, wherein the apparatus is reusable.

14. The electromagnetic medical device instrument tracking apparatus of claim 13, wherein the apparatus is sanitizable and disinfectable.

15. A method, comprising:
selecting a medical device for a procedure, wherein the medical device includes an elongated shaft carrying an instrument at a proximal end;
selecting EM medical device instrument tracking apparatus corresponding to the selected medical device, the EM medical device instrument tracking apparatus including:
an elongated hollow channel, including:
a cylindrically shaped body configured to receive elongated shafts of medical devices having instruments at ends of the elongated shafts;
a distal end;
an inlet at the distal end;
a proximal end; and
an outlet at proximal end;
a fastener coupled at the distal end;
at least one electromagnetic sensor disposed at the proximal end; and
an electrical pathway in electrical communication with the at least one
electromagnetic sensor and configured to route signals from the at least one electromagnetic sensor off the apparatus;
installing the selected medical device in the EM medical device instrument tracking apparatus,
wherein the cylindrically shaped body positions the electromagnetic sensor by an instrument located at an end of an elongated shaft of a medical device installed in the cylindrically shaped body;
fastening the selected medical device and the EM medical device instrument tracking apparatus together; and
connecting the electrical pathway of the EM medical device instrument tracking apparatus to the tracking system.

16. The method of claim 15, wherein the cylindrically shaped body has a first length, the elongated shaft has a second length, and the first length corresponds to the second length.

17. The method of claim 15, wherein the cylindrically shaped body has a first diameter, the elongated shaft has a second diameter, and the first diameter corresponds to the second diameter.

18. The method of claim 15, further comprising:
employing the EM medical device instrument tracking apparatus to track a spatial orientation of the medical device with respect to the tissue of interest.

19. The method of claim 15, further comprising:
uninstalling the selected medical device from the EM medical device instrument tracking apparatus.

20. An electromagnetic medical device instrument tracking apparatus, comprising:
an elongated hollow channel;
at least one electromagnetic sensor disposed at a proximal end of the elongated hollow channel; and
an electrical pathway in electrical communication with the at least one electromagnetic sensor.
